(19) United States
(12) Patent Application Publication
(10) Pub. No.: US 2022/0049269 A1
(43) Pub. Date: Feb. 17, 2022

(54) COMPOSITIONS AND METHODS FOR THE IN SITU DELIVERY OF THERAPEUTIC AND DIAGNOSTIC AGENTS

(71) Applicant: SynLife, Inc., Cambridge, MA (US)

(72) Inventors: Felix MOSER, Cambridge, MA (US); Katarzyna ADAMALA, Minneapolis, MN (US); David BENJAMIN, Cambridge, MA (US); Edward Stuart BOYDEN, Chestnut Hill, MA (US); Jeremy WERTHEIMER, Brookline, MA (US); Kevin MUNNELLY, Cambridge, MA (US); Elazer EDELMAN, Brookline, MA (US)

(73) Assignee: SynLife, Inc., Cambridge, MA (US)

(21) Appl. No.: 17/279,069

(22) PCT Filed: Sep. 23, 2019
(86) PCT No.: PCT/US2019/052467

§ 371 Date: Mar. 23, 2021

Related U.S. Application Data
(60) Provisional application No. 62/854,617, filed on May 30, 2019.
(60) Provisional application No. 62/735,608, filed on Sep. 24, 2018.

(51) Int. Cl. C12N 15/85

(52) U.S. Cl. CPC C12N 15/85

(57) ABSTRACT

Synthetic minimal cells (SMC) are provided for delivering a therapeutic or diagnostic agent to a subject or to a site within a subject in need of treatment or diagnosis. SMCs may be targeted to the site or sense the site and initiate production and release of the therapeutic or diagnostic agent. SMCs comprise a sensor, at least one genetic circuit and outputting means to deliver the therapeutic agent.

### BACKGROUND OF THE INVENTION

Targeted drug delivery, sometimes called smart drug delivery, is a method of delivering medication to a patient in a manner that increases the concentration of the medication in some parts of the body relative to others. This means of delivery is largely founded on nanomedicine, which employs nanoparticle-mediated drug delivery in order to overcome certain disadvantages of conventional drug delivery. Nanoparticles would be loaded with drugs and targeted to specific parts of the body where there is for example diseased tissue, thereby avoiding interaction with healthy tissue. The goal of a targeted drug delivery system is to prolong, localize, target and have a protected drug interaction with the diseased tissue.

Despite the targeted nature of this technology, the process of delivery is not based on or responsive to the presence of certain local signals to identify where and under what conditions the drug should or should not be released. A smarter drug delivery system would include detection then response. Such a system would offer better targeting and more precise delivery. Need exists for such delivery methods, for therapeutic as well as diagnostic purposes.

### BRIEF SUMMARY OF THE INVENTION

In one embodiment, the invention is directed to a synthetic minimal cell (SMC) or a consortium of SMCs for the production and delivery of a therapeutic or diagnostic agent comprising:
(a) at least one sensor that detects at least one condition under which the therapeutic or diagnostic agent is to be produced and delivered;
(b) at least one genetic circuit that controls the production of the therapeutic or diagnostic agent upon detection of the at least one condition by the sensor; and
(c) at least one outputting means for delivering the therapeutic or diagnostic agent outside of the SMC.

In one embodiment, the invention is directed to a synthetic minimal cell (SMC) or a consortium of SMCs for the delivery of a therapeutic or diagnostic agent, wherein each SMC comprises:
(a) at least one therapeutic or diagnostic agent;
(b) at least one sensor that detects at least one condition under which the therapeutic or diagnostic agent is to be delivered; and
(b) at least one genetic circuit that controls the production of an outputting agent for delivering the at least one therapeutic or diagnostic agent outside the SMC upon detection of the at least one condition by the sensor.

In one embodiment, the therapeutic, diagnostic or outputting agent is a protein, peptide, nucleic acid or small molecule. In one embodiment the nucleic acid is DNA, RNA, shRNA, siRNA, an antisense oligonucleotide, a microRNA inhibitor, an anti-miRNA or a sgRNA. In one embodiment, output of the SMC is an antibody or antibody fragment, such as Fab or scFv.

In one embodiment, the therapeutic, diagnostic or outputting agent is not produced until the genetic circuit activates production of the therapeutic agent or a precursor thereof within the SMC.

In one embodiment, the sensor is membrane-bound or soluble within the SMC. In one embodiment, the sensor detects one or more conditions that exist at a site where delivery of the therapeutic agent is desired. In one embodiment, the one or more conditions exist at the site is a result of the disease to which the therapeutic or diagnostic agent is desirously delivered, such as but not limited to the presence of one or more biomarkers, cell surface receptors, metabolites, microenvironmental markers, peptides, nucleic acids or fragments thereof, or proteins or fragments thereof. In one embodiment, the one or more conditions are provided at the site where the therapeutic agent is beneficially delivered. In one embodiment, the one or more conditions are selected from radiation (such as ionizing, infrared, visible, ultraviolet and any other electromagnetic radiation), heat, pH change, or the administration of an agent that targets the site desirous of beneficial therapeutic or diagnostic agent release and activates the SMC sensor, production, or outputting of the therapeutic or diagnostic agent, or any combination thereof.

In one embodiment, the therapeutic or diagnostic agent production comprises translation.

In one embodiment the therapeutic or diagnostic agent production comprises gene expression. In one embodiment, the production is controlled by transcription factors. In one embodiment the therapeutic or diagnostic agent production comprises a multi-enzyme biosynthetic pathway.

In one embodiment, the outputting means is by passive or controlled release from the SMC. In one embodiment, the outputting means is by physicochemical changes in the SMC that permit passive release of a therapeutic agent. In one embodiment the outputting means is responsive to a condition different from that of the sensor. In one embodiment the outputting means further comprises a negative feedback sensor that reduces production or outputting of the therapeutic or diagnostic agent.

In one embodiment, the outputting agent is a protein, a peptide, a small molecule, a membrane channel polypeptide (also referred to herein as a “pore”), a membrane pump polypeptide, a trafficking polypeptide, a signal polypeptide or an export polypeptide.

In one embodiment, the sensor, production and outputting means of the therapeutic or diagnostic agent occurs in one, or two or three different SMCs. In one embodiment, the delivery of the therapeutic or diagnostic agent occurs cooperatively among multiple SMCs.

In one embodiment, methods are provided for treating a condition or disease benefitted by a therapeutic agent comprising administering to the subject in need thereof or to a site in said subject a SMC as described herein above. In one embodiment the SMC is targeted to the site benefitted by the delivery of the therapeutic agent. In one embodiment the subject is administered an agent that targets the site to which the SMCs of the invention are then targeted to and activated.

In one embodiment, methods are provided for diagnosing a condition or disease comprising administering to the subject in need thereof or to a site in said subject a SMC as described herein above, wherein the SMC releases a diagnostic agent. In one embodiment , the diagnostic agent is detected in a bodily fluid of the subject, such as blood or urine. In one embodiment the diagnostic agent is detectable by imaging.

In one embodiment, a consortium of SMCs are used for therapeutic or diagnostic purposes, the consortium comprising a plurality of different, sensor-specific SMCs each responsive to a different pathogen or condition, wherein the pathogen or condition activates production by the SMCs specific for that pathogen or condition, wherein a therapeutic agent or diagnostic agent specific for that pathogen or condition is released by the SMC.

These and other aspects of the invention will be appreciated from the description of the drawings and a detailed description of the invention.

### BRIEF DESCRIPTION OF THE FIGURES

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts the overall concept of the invention using SMCs as programmable therapeutic agents;

FIG. 2 depicts some of the potential uses of the SMCs of the invention for sensing of and responding to diseases tissue by producing and releasing a therapeutic agent;

FIG. 3 depicts SMCs responsive to embolisms by producing a thrombolytic agent on-site; and

FIG. 4 depicts SMCs can be programmed to respond to specific combinations of cancer metabolite to generate chemotherapeutic molecules.

FIG. 5A, FIG. 5B and FIG. 5C depict an SMC that senses a particular bacterial species or type and activates (A) the production of an antibiotic or antimicrobial agent specific for that bacterial species or type; (B) the production of a pore or other membrane-lysing agent that allows release of a bacterial species- or type-specific antibiotic or antimicrobial agent present in the SMC; or (C) the production of a reporter or marker of that bacterial species or type, the reporter or marker than can be identified in a bodily fluid sample from the subject and indicate the appropriate therapeutic regimen to treat that bacterial species or type.

FIG. 6A and FIG. 6B depicts a scheme by which SMCs comprising nested liposomes containing the chemotherapeutic doxorubicin and circuitry expressing blue light-sensitive split T7 RNA polymerase. The T7 promoter in this system expresses an alpha-haemolysin (aHL) pore complex that releases doxorubicin from the nested liposomes and SMC upon activation of split T7 with blue light. FIG. 6B shows data demonstrating that split T7 systems function in cell-free systems, including a rapamycin-sensitive system (Rapa-T7) and a blue light-sensitive system (Opto-T7). Here, the output T7 promoter generates a GFP protein the fluorescence of which can be measured. The unsplit T7 (“Whole T7 Control”) and Split T7 system without sensing domains (“Split T7 Control”) are shown as controls.

FIG. 7 shows expression of GFP and a tyrosinase (melA) in cell-free systems. Under white light (top), a brown pigment (melanin) is produced when the melA gene, L-tyrosine, and copper ions are present. Under blue light (bottom), GFP production is observed as a control. Melanin is a brown pigment that can absorb broad-spectrum light and generate heat as a result. Such a system could be useful in using SMCs to conduct heat ablation of target tissue.

FIG. 8 shows results of a viability assay when cell-free systems generating different cytotoxic proteins are added to tissue cultures of HEK293 cells. Here, the PURE cell-free system containing disulfide bond enhancer (DBE) is used. When SMCs expressing alpha-haemolysin are added to the culture, most cells are killed. No toxicity is observed from any of the other proteins. This demonstrates how SMCs could generate different proteins with specific toxic effects.

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” or “medicament,” are used interchangeably herein, as context dictates, to refer to a compound or compounds or composition of matter which, when administered to a subject (human or animal) induces a desired pharmacological and/or physiologic effect by local and/or systemic action. A personalized composition or method refers to a product or use of the product in a regimen tailored or individualized to meet specific needs identified or contemplated in the subject.

The terms “subject,” “individual,” and “patient” are used interchangeably herein, and refer to an animal, for example a human, to whom treatment with a composition or formulation in accordance with the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The compositions described herein can be used to treat any suitable mammal, including primates, such as monkeys and humans, horses, cows, cats, dogs, rabbits, and rodents such as rats and mice. In one embodiment, the mammal to be treated is human. The human can be any human of any age. In an embodiment, the human is an adult. In another embodiment, the human is a child. The human can be male, female, pregnant, middle-aged, adolescent, or elderly. According to any of the methods of the present invention and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine or porcine. In another embodiment, the subject is mammalian.

Conditions and disorders in a subject for which a particular drug, compound, composition, formulation (or combination thereof) is said herein to be “indicated” are not restricted to conditions and disorders for which that drug or compound or composition or formulation has been expressly approved by a regulatory authority, but also include other conditions and disorders known or reasonably believed by a physician or other health or nutritional practitioner to be amenable to treatment with that drug or compound or composition or formulation or combination thereof.

The inventors herein have discovered a novel means of programming and employing synthetic minimal cells (SMCs) as a means of delivering therapeutic molecules. SMCs, much like living cells, are composed of a phospholipid bilayer (the membrane of a liposome) that encapsulates a mixture of macromolecules and reagents that is capable of DNA translation or gene expression. As described in detail below, SMCs can be programmed with genetic circuits to enable complex molecular computation. SMCs have several unique capabilities that make them a valuable platform for drug delivery. In one embodiment, SMCs have low toxicity: liposomes are already used as vehicles in several medications and can be shielded from the immune system. In one embodiment SMCS have versatility: SMC membranes and components can be formulated in a number of ways to adapt them to specific clinical needs. In one embodiment, SMCs have programmability: SMCs can be genetically programmed to sense a variety of conditions and conditionally generate RNA, protein or other molecules with diagnostic or therapeutic value. In one embodiment, SMCs have adaptability: SMCs sense-and-respond systems can conditionally control how much therapeutic molecule is made and for how long, thereby preventing over-production and improving safety. In the descriptions herein, any one or more of the foregoing embodiments are incorporated into SMCs of the invention.

The foregoing capabilities make SMCs a unique formulation that has the potential to be a platform technology for smart therapeutics that can address a wide variety of medical needs. In one embodiment, these capabilities enable delivery of current drugs in a way that safer and more efficacious, since it would allow more specific, on-site deployment of the drug.

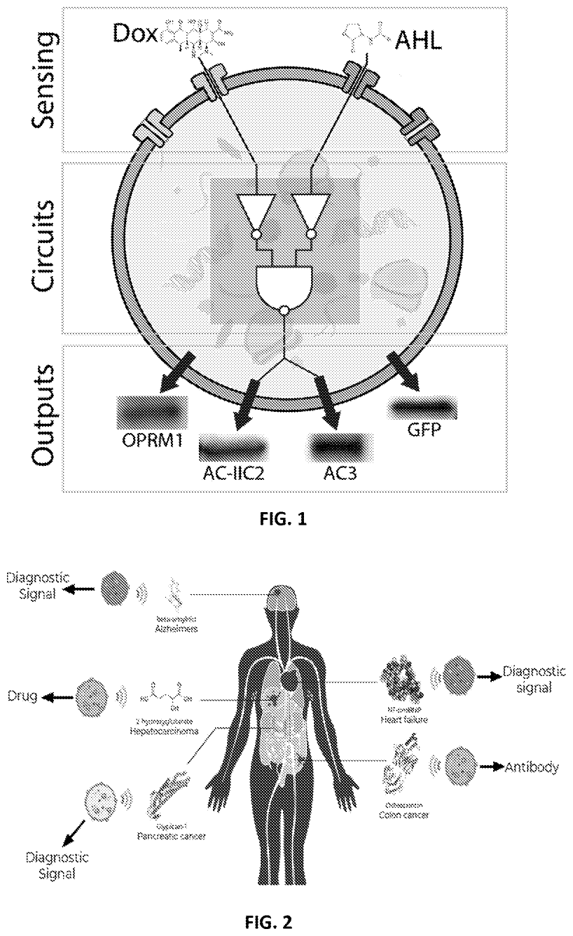

In one embodiment, SMCs comprising at least three modules that together control their behavior (FIG. 1):
1. Sensing. Using natural protein or RNA-based sensor systems that are either integrated in the membrane or soluble within the SMC, these modules enable the SMC to sense changes in its environment. These changes include physical (e.g. temperature) as well as chemical (e.g. pH, specific molecules) changes and can be used to detect the environment around specific tissue types (e.g. blood clots, tumors). These changes include the presence of a receptor, cell surface marker, metabolite, microenvironmental marker or other cellular component or combinations thereof that activates the sensor and results in the delivery of the therapeutic agent to the site by the SMC. The SMC membrane lipid composition or other components therein may be sensitive to the detected condition. These one or more conditions or molecules that are detected by the sensor may be referred to herein as activators. As will be noted below, the requirement for two or more activators to activate the SMC may be utilized to optimize delivery to a specific target.
2. Circuits. Using protein transcription factors and other means, sophisticated genetic circuits can be engineered that control the information flow within the SMC. This essentially controls which combination of sensed inputs control which outputs. The circuits are more fully described below.
3. Outputs. The output of the system is either one or more proteins, nucleic acids, fragments or components thereof or a small molecule, which is generated only once certain conditions are met, as determined by the sensors and circuitry. Exemplary conditions include SMC localization to a specific tissue or the presence of a particular marker molecule, such as fibrin identifying a clot, tumor cell surface markers identifying a tumor, and increased expression of any cell surface marker or metabolite that indicates a disease condition in the cell or tissue. Other markers include microenvironmental markers such as a change in oxygen tension or pH. Possible proteins generated include but are not limited to antibodies, toxins, hormones, markers, and enzymes.

Proteins can serve as diagnostic markers or deliver therapeutic value to the targeted tissue. RNA that is generated can serve as a therapeutic moiety (e.g. siRNA) or as a diagnostic marker that can later be assessed by sequencing. Subcomponents that self-assemble (or require an agent to assemble, such as multimeric protein complexes) into a therapeutic agent are also embraced herein.

As noted herein, in one embodiment of the invention, the SMCs of the invention comprise a therapeutic or diagnostic agent, and the sensor and genetic circuit facilitates the delivery of the SMC contents. Thus, the SMC does not necessarily generate the therapeutic or diagnostic agent from precursors within the SMC (or metabolites within the in vivo milieu) but the genetic circuit enables the release of the content of the SMC. In some embodiments a SMC may comprise both a therapeutic and diagnostic agent and the genetic circuit generates and additional agent, as well as means for delivery of the SMC contents.

In one embodiment, a consortium of SMCs are used for therapeutic or diagnostic purposes, the consortium comprising a plurality of different, sensor-specific SMCs each responsive to a different pathogen or condition, wherein the pathogen or condition activates production by the SMCs specific for that pathogen or condition, wherein a therapeutic agent or diagnostic agent specific for that pathogen or condition is released by the SMC.

In one embodiment, a consortium of SMCs are used for therapeutic or diagnostic purposes, the consortium comprising a plurality of different, sensor-specific SMCs each responsive to a different pathogen or condition, wherein the pathogen or condition activates production of a therapeutic or diagnostic agent by the SMCs specific for that pathogen or condition, wherein the therapeutic agent or diagnostic agent specific for that pathogen or condition is released by the SMC. In one embodiment, the therapeutic agent or diagnostic agent is present within the SMC and activation of the SMC results in the release or delivery of the therapeutic or diagnostic agent from the pathogen or condition specific SMCs.

In one embodiment, a population of different types of SMCs are used together for certain purposes. In one non-limiting example, the population of SMCs includes different types of SMCs each capable of sensing an individual species of bacteria or type of bacteria, each such type of SMC capable or releasing a bacterial species- or type-specific therapeutic, or diagnostic agent, in the presence of that bacterial species or type. For example, different bacterial species include [Staphylococcus aureus, Streptococcus pyogenes, Pseudomonas aeruginosa, Campylobacter jejuni, Corynebacterium diphtheriae, Helicobacter pylori, Legionella pneumophila, Propionibacterium acnes, Mycobacterium tuberculosis, Escherichia coli, Klebsiella sp., Neisseria sp., etc.), by way of non-limiting example. For example, different bacterial types include [Gram negative, Gram positive, actinomycetes, cyanobacteria, spirochaetae, firmicutes, proteobacteria, chlamydiae, etc.), by way of non-limiting example. This population of SMCs for the purposes herein is called a consortium of SMCs. Consortia of SMCs may be prepared to address bacterial infections broadly, or may comprise subsets of different SMCs to treat or diagnose an infection based on certain already-established characteristics that rule in or rule out certain bacterial species or types. In one non-limiting example, the patient has pneumonia or unknown origin; the SMC consortium comprises SMCs that sense different bacterial species known to cause pneumonia. As noted herein, such SMCs upon sensing the particular bacterial species or type, may (A) activate a genetic circuit to produce then release an antimicrobial product; (B) activate a genetic circuit to produce then release a reporter or marker that can be detected in the patient's bodily fluid indicating the type of bacterial infection; (C) activate a genetic circuit that allows the release of an antimicrobial product or a diagnostic reporter or marker contained within the SMC; or (D) activates a genetic circuit that produce a product that triggers other SMCs to perform any one or more of the foregoing functions.

Each of the foregoing aspects of the SMCs of the invention are described below, with examples that are intended to be exemplary and non-limiting as to the breadth of the invention. Each example is an embodiment of the invention.

### 1. SMC Components

SMCs are liposomes that encapsulate one or more macromolecules that can modulate the physicochemical composition or behavior of the SMC in response to some physicochemical input. Liposome encapsulation of synthetic minimal cells (SMCs) enables chemical reactions to proceed in well-isolated, molecularly crowded environments. SMCs include compartmentalized genetic circuits or cascades. As used herein the term “genetic circuit” refers to a set of chemicals, one part of which triggers the initiation, modulation or otherwise alters generation of a gene product, which then can directly or indirectly initiate, modulate, or otherwise alter the generation of another gene product encoded for by another part of the genetic circuit. The use of genetic circuits permits scaling of production (a non-limiting example of which is gene expression for polypeptide production) and permits low, moderate, and/or high levels of complexity in the production process, which may be determined by the engineering of the SMCs and genetic circuits of the invention. Certain SMCs of the invention are prepared such that they contain genetic cascades that can be triggered, modulated, reduced, or induced by one or more of an internal stimulus or an external chemical stimulus. Some aspects of the invention include preparation and/or use of populations of SMCs that are able to operate genetic cascades in parallel to one another and/or to jointly regulate their cascades via exchanged small molecule messengers. The terms: “liposome”, “synell”, and “synthetic minimal cell” (SMC) are used interchangeably herein in reference to liposome bioreactors performing some of the biochemical functions of the living cell, most notably transcription or translation for the expression of nucleic acids and proteins of therapeutic interest and utility.

Methods and compositions of the invention, in some aspects, permit modularity of multi-component genetic circuits and cascades in synthetic biology. By encapsulating genetic circuits and cascades within SMCs and orchestrating the SMCs to either operate in parallel, communicate with one another, or fuse with one another in a controlled way, methods of the invention can be used to create and utilize genetic cascades that take advantage of the modularity enabled by liposomal compartmentalization. Thus, in some aspects of the invention, methods are provided that enable genetic cascades to proceed in well-isolated environments while permitting the desired degree of control and communication. SMCs of the invention may be used singly, in combination with other SMCs, in networks of other SMCs, or in other conformations with other SMCs that support complex chemical reactions that benefit from both the high-fidelity isolation of multiple reactions from one another, as well as controlled communication and regulatory signal exchange between those reactions. Such regulation can be put to therapeutic use in identifying and controlling the delivery of a therapeutically useful molecule at the site most needed or most efficacious, with the least side effects, in a subject in need of therapy. Such regulation can also be put to diagnostic use by identifying and controlling the delivery of a reporter or marker that can be identified for example in a bodily fluid such as blood or urine of the subject, to aid in diagnosing the disease and directing specific treatment. In one example, a biosynthetic pathway may be segregated into different SMCs if intermediates in the pathway might interfere with the efficiency of an earlier or later step.

Compositions have been prepared that permit maximization of the modularity of their design to enable the integration of different reaction networks and to optimize their scalability and flexibility, applicable to the targeted delivery of therapeutically useful agents. One aspect of the invention includes methods of encapsulation of genetic circuits and reaction cascades within SMCs thereby permitting chemical reactions to proceed in well-isolated environments. It has been demonstrated that it is possible to engineer genetic circuit-containing SMCs to contain multiple-part genetic cascades, and that these cascades can be controlled by external signals as well as inter-liposomal communication without cross-talk. Methods of the invention have now been demonstrated that result in SMCs containing different cascades to be fused in a controlled way so that the products of incompatible reactions can be brought together; such methods are adaptable to delivery of therapeutic agents and the control of the duration and extent of delivery. In some aspect of the invention, compositions are provided that include one or more SMCs. Methods of the invention, in some embodiments include use of such SMCs to enable more modular creation of synthetic biology cascades, an essential step towards their programmability, and utility for delivering a therapeutic agent to a site of interest.

Certain aspects of the invention include a SMC that includes at least a portion of at least one multi-gene genetic circuit. A portion of a multi-gene genetic circuit may be part of a multi-gene genetic circuit that is present in one SMC and part of the multi-gene genetic circuit that is present in another SMC. In certain aspects of the invention, less than a full multi-gene genetic circuit may be present in an SMC of the invention and the remainder of the full multi-gene circuit may be present in one additional SMC. In another non-limiting example, in certain aspects of the invention, less than a full multi-gene genetic circuit (also referred to herein as “a portion”) may be present in an SMC of the invention and another part of the multi-gene genetic circuit may be present in one additional SMC, and a further part of the multi-gene genetic circuit may be present in another additional SMC, etc. Thus, a multi-gene genetic circuit of the invention may include genes that are expressed in different SMCs of the invention, for example, an SMC of the invention may include one or more genes of a multi-gene genetic circuit and a second SMC of the invention may include one or more independently selected genes of the same multi-gene genetic circuit and one of the SMCs may express a polypeptide that directly or indirectly induces expression of a polypeptide in another SMC. Thus, two or more SMCs may be part of the same multi-gene genetic circuit. In certain aspects of the invention, an SMC may include all of the genes that make up a multi-gene genetic circuit. A multi-gene genetic circuit may include 2, 3, 4, or more genes, which are also referred to herein as “gene components” of the multi-gene genetic circuit. The utility of such multi-gene circuits in sensing and delivering therapeutic agents will be evident in the examples provided herein.

Some aspects of the invention include methods of preparing SMCs of the invention, and methods of their use. As used herein the term “multi-gene genetic circuit” means two or more genes that interact either directly or indirectly with each other. For example, a polypeptide expressed by a gene in a vector in an SMC of the invention may trigger, modulate, reduce, or induce expression of one or more of a second, third, fourth, fifth or more genes in the SMC and/or in another SMC, with the goal to regulate the delivery of a therapeutic agent. The presence of a circuit indicates that expression activity of one gene modulates expression of another gene in one or more of the same or another SMC of the invention.

Expression of a gene of a multi-gene genetic circuit is also referred to as an “activity” of the gene. Contacting an SMC of the invention with an agent may modulate (increase or decrease) an activity of a gene that is part of a multi-gene genetic circuit. In certain instances, an agent that modulates activity of a gene is an exogenous agent that is contacted with the SMC. As noted herein, such exogenous agent may be a compound or cell surface protein or other factor in the proximity of diseased tissue, such as a tumor, to which SMCs of the invention will be targeted in order to deliver the therapeutic compound at the tumor site. In other embodiments, a diagnostic agent may be released that is detectable for example in a bodily fluid, identifying the disease and directing appropriate therapy. In other embodiments, the site may be demarcated by external stimuli, such as application of heat, electromagnetic or ionizing radiation, or another physico-chemical change that activates the production of the therapeutic agent at the site. In another embodiment, an agent can be administered to the subject that targets the desired site of SMCs such that SMCs at that site produce the desired therapeutic agent. The foregoing descriptions are meant to be exemplary only and non-limiting as to the aspects of the invention.

An exogenous agent may be added to the external environment of an SMC from a source external to the SMC's environment, or may be produced or released by another SMC that is present in the SMC' s environment. In some instances, an agent that modulates activity of a gene is an endogenous agent that is expressed within an SMC and the expressed agent modulates an activity of another gene in that SMC. In certain aspects of the invention, a gene that is part of multi-gene genetic circuit may express a polypeptide in an SMC and the polypeptide alters the internal environment of the SMC, thus modulating expression of another gene component of the multi-gene genetic circuit in the SMC. For example, though not intended to be limiting, a gene in an SMC may encode a channel protein that is expressed in the SMC and permits entry of an agent such as a small molecule, etc. that modulates (for example, increases or decreases) expression of another gene in the multi-gene genetic circuit of the SMC. As used herein the term “externally delivered” used in relation to an agent, means an agent that is an exogenous agent. In some aspects of the invention, the agent is a small molecule and in certain embodiments, the agent is soluble.

As used herein, the product of a genetic circuit in a SMC of the invention can be a protein or peptide, a nucleic acid such as a siRNA, or a small molecule that is a product of a biosynthetic pathway at least one step thereof is modulated by a genetic circuit in the SMC. As will be illustrated in more detail below, the production of a therapeutically beneficial small molecule from one or more precursors within the SMC (which precursors are not therapeutically beneficial) is an embodiment of the invention.

As used herein, the terms “increases” or “increase” in reference to expression of a nucleic acid or polypeptide means raising a level of expression from zero to any amount above zero or raising the level of expression from an existing level to a higher level of expression. As used herein, the terms “decreasing” or “decrease” in reference to expression of a polypeptide, nucleic acid or small molecule means lowering a level of expression from an amount to an amount that is lower, which may be, but need not be a level of zero expression.

Certain aspects of the invention include SMCs having one or more functional characteristics, non-limiting examples of which include: expression of one or more nucleic acids or polypeptides; triggering expression of one or more nucleic acids or polypeptides internal to the SMC; triggering expression of one or more nucleic acids or polypeptides external to the SMC, for example in one or more additional SMCs; modulation of an activity of a polypeptide internal to the SMC to reduce expression of its encoded polypeptide; modulation of an activity of a polypeptide present in another SMC to reduce expression of its encoded polypeptide; communication with one or more additional SMCs or with other elements external to the SMC; etc. In some aspects of the invention, modulating an activity comprises increasing the activity and in certain embodiments of the invention modulating an activity comprises the decreasing activity. Additional examples of functional characteristics that may be present in SMCs of the invention are described herein. As noted above, any activity of a polypeptide may be activity of a biosynthetic pathway utilizing that polypeptide (e.g., an enzyme) and thus are embodiments of the invention.

Certain aspects of the invention include SMCs that have one or more structural characteristics, non-limiting examples of which include: liposomal encapsulation; inclusion of one, two, three, four, or more expression vectors; an internal environment suitable for transcription, or transcription and translation, of one or more genes; one, two, three, four, or more genes that can be triggered or induced to express their encoded polypeptide or modulated to reduce expression of their encoded polypeptide; one or more expression vectors that encode fusion proteins; encoded detectable labels; decoration of the external liposomal surface with one or more of a detectable label, a fusion molecule, a delivery molecule, etc. Additional examples of structural characteristics that may be present in SMCs of the invention are described herein. Some aspects of the invention also include methods of preparing SMCs of the invention, and methods of their use. Moreover, production of nucleic acid molecules such as mRNA, shRNA, siRNA, among others, is an embodiment of the invention; translation of mRNA into protein is not necessary in the production of therapeutically useful nucleic acid agents.

SMCs and methods of their use as encompassed by the invention allow SMCs containing genetic circuits to be regulated externally, to communicate with each other, and to work together in networks. Such networks may encompass the means to deliver a desired therapeutic agent at a particular site in the body, relying on a combination of signals from the body such as expressed tumor antigens and tumor metabolites, that in combination, signal the production of the therapeutic agent only when all of the signaling molecules are present; this, a highly targeted therapeutic approach to treatment of disease, and a fine-tuned delivery of therapeutically effective molecules only under certain circumstances, supports the concept of “personalized SMCs” that can be provided to treat a particular disease. In one non-limiting example, the SMCs are a combination of a tumor-specific SMC that senses a particular tumor marker and produces a molecule which is a signal for production of a toxin by another SMC that is not sensitive to the particular biomarker. In one embodiment, personalized and general SMCs can be combined to tailor the therapy for a particular individual. In a further embodiment, the tumor-specific SMC can also have multiple circuits that can determine among several triggers which biomolecule to produce; that biomolecule can then trigger one among a mixture of toxin-producing SMCs to produce the particular toxin most appropriate for a tumor expressing the biomarker. As noted elsewhere herein, combinations of different SMCs each triggered by a different agent, may be provided when for example the specific infectious agent is not known, such that the subset of SMCs in the combination that is triggered by the specific pathogen induces the production and release of a therapeutic agent specific for that pathogen, or in another embodiment, releases a diagnostic agent that is detectable in the patient's urine or blood that indicates the type of pathogen thus informing specific treatment. These examples are not intended to be limiting as to the potential combinations of SMCs that can be used therapeutically or diagnostically for precise medical treatment.

A non-limiting example of a benefit of an SMC of the invention is in its use to provide modularity in synthetic biology procedures and methods. An additional non-limiting example of methods of use of SMCs of the invention is in studies on the pathophysiology of the disease process, by generating signals from SMCs that can be used to, for example, trace the progression of disease. Other non-limiting examples are for diagnostic purposes, where, for example, SMCs sensing a particular site of disease will generate a detectable marker that can be use by the health care provider to target or personalize the treatment depending on the location, or the detectable marker identifiable in the patient's blood or urine that is diagnostically useful for informing treatment. Additional methods for which SMCs of the invention may be used will be recognized by those skilled in the art.

Certain aspects of the invention comprise encapsulating cell-free transcription/translation (also referred to as “TX/TL”) extracts or transcription (“TX”) extracts into liposomes to create bioreactors, which are referred to herein as SMCs. TX/TL components may be prokaryotic or eukaryotic. Means to prepare single gene SMCs, and the use of cell-free TX/TL extracts in artificial cells and liposomes are known in the art, see for example: Zemella, A et al., (2015) ChemBiochem Vol. 16, Issue 17:2420-2431; Forster, AC. & Church, G.M, (2006) Mol. Syst. Boil 2, 45; Brea, R. J. et al., (2015) Chem. A Eur. J. Vol. 21, Issue 36:12564-12570; Luisi, P. L. et al., (2006) Naturwissenchaften 93, 1-13; Stano, P. & Luisi, P. L. Curr Opin Biotechnol. (2013) 24:633-638; Tan, C. et al. (2013) Nat. Nanotechnol. 8, 602-8; de Souza, T. P. et al. (2012) Orig. Life Evol. Biosph. 42, 421-428; de Souza, T. P., et al., (2014) J. Mol. Evol. 79, 179-192; and Caschera, f. & Noireauz, V. (2014) Curr. Opin. Chem. Biol. 22, 85-91, each of which is incorporated herein by reference in its entirety. SMCs of the invention which include multiple genes can be prepared using methods presented herein in conjunction with routine procedures known in the art. Methods and components for liposomal encapsulation are known in the art and can be used in the preparation of SMCs of the invention.

SMCs of the invention can be used to make functional proteins or other therapeutically useful molecules using encapsulated systems reconstituted from recombinant cell-free translation factors and/or cell-free extracts from bacterial and/or eukaryotic cells. Unlike previous liposomal SMCs, which were used to express single genes and to synthesize a single gene product within a homogenous population of SMCs, certain embodiments of the present invention include SMCs that comprise multi-component genetic circuits, for example two, three, four, five, or more different genes that synthesize two, three, four, five or more different gene products, respectively. In addition, certain embodiments of the invention include preparation and use of SMCs that include multi-component circuits that can operate across multiple well-compartmentalized SMCs. The invention, in some aspects, includes strategies for constructing and utilizing such networks of SMC-based genetic circuits, thus expanding the control and amplification capacities of SMCs. In one embodiment, a biosynthetic pathway to produce a therapeutically useful agent comprises multiple enzymes, each of which is produced in the SMC upon activation.

Engineered networks of SMCs of the invention can be used to support complex chemical reactions that benefit from both the high-fidelity isolation of multiple reactions from one another, as well as controlled communication and regulatory signal exchange between those reactions. As noted above these are particularly useful in the preparation of therapies for diseases in which the targeted delivery of a particular agent depends on the microenvironment at the site of disease in the body, and other means of identifying the best therapy cannot be made empirically. In one embodiment, the combination of SMCs is capable of sensing the particular conditions and producing and delivering the appropriate therapeutic agent, or detectable diagnostic agent. As will be noted herein, the therapeutic or diagnostic agent may be one or more small molecule compounds, peptides, proteins or nucleic acids, by way of non-limiting example.

Cascade circuits of the invention, in which the product of one gene triggers the production of the next, are useful for a variety of reasons-for signal amplification (i.e., a relatively small input signal can trigger a high output), for modularity (e.g., a variety of sensors can be connected to a given output), and to enable multi-node control at various points within the network (as in the configuration of natural signaling and metabolic pathways in cells), where many reagents must be regulated in timing and concentration, for efficient synthesis. In some aspects of the invention, two or more SMCs that operate in conjunction with each other are also referred to herein as a “network” of SMCs. As used herein the term “network” used in conjunction with SMCs means two or more SMCs that interact with each other and can function as a system. A non-limiting example of a means by which two or more SMCs interact is chemical communication between SMCs. For example, though not intended to be limiting, as at least part of a network of SMCs, a first SMC releases an agent that contacts a second SMC and acts as a signal that triggers an action in the second SMC. Further to the foregoing example, in some aspects of the invention, after receiving the signal from a first SMC, a second SMC then release a signal that triggers an action in one or more of the first SMC, a third, fourth, or other SMC. In some embodiments of the invention, communication between two SMCs is one-directional communication and in certain embodiments of the invention communication between two SMCs is bi-directional communication. As noted above, such triggers and cascades can be provided to personalize the therapeutic agent to be delivered to the site. In another embodiment, the level of optimal expression or synthesis of the therapeutic agent for the particular disease is sensed by SMCs and through the same or a network of SMCs converted into the appropriate amount of therapeutic agent to deliver to the site. In another embodiment, the duration of treatment is programmed into the genetic circuits to deliver, for example, pulses of therapeutic agent interrupted by periods of no delivery, or alternate deliver of different therapeutic agents at different times, for optimal therapy of a particular disease. In one embodiment, the temporal, local delivery of two or more therapeutic agents can be provided by appropriate SMCs.

As used herein in reference to gene expression, the term “cascade” means triggering (also referred to herein as “inducing”) two or more events by an agent. In certain aspects of the invention, a triggered event may be expression of a polypeptide, nucleic acid or small molecule in one or more SMCs. For example, though not intended to be limiting, a small molecule may contact an SMC of the invention and trigger expression of one or more polypeptides from genes contained in the SMC, or other small molecules that are products of a biosynthetic pathway utilizing the polypeptides.

The one or more polypeptides may in turn induce expression of one or more additional polypeptides within the SMC or within a second SMC or a plurality of SMCs. In some aspects of the invention, a cascade amplifies expression of at least one polypeptide in at least one of a first SMC, a second SMC, or a plurality of SMCs. In another non-limiting example of a cascade, a polypeptide comprising a membrane channel or membrane pump may be expressed in an SMC of the invention and following that expression, the channel permits passage (entry and/or exit) of agents such as small molecules, polypeptides, ions, etc. into or out of the SMC. The agents may then trigger additional expression in the SMC or in a second or a plurality of SMCs that are contacted by the agent(s) that passed through the expressed channel. Non-limiting examples of polypeptides comprising membrane channels and polypeptides that comprise membrane pumps are light-activated ion channels and light-activated ion pumps, respectively. Light-activated ion channels polypeptides and light-activated ion pump polypeptides suitable for use in methods and compositions of the invention are known in the art. Methods suitable to prepare and use expression vectors, polynucleotide sequences, promoters, delivery agents, labeling agents, etc. to express polypeptides in SMCs of the invention are known in the art. Any of the foregoing embodiments are equally applicable to small molecules.

In addition to preparing and using multi-component genetic circuits that are encapsulated within SMCs, the invention in some aspects, also includes created systems in which specific circuit elements are compartmentalized within different sets of SMCs within the same external solution. Such compartmentalization can serve key purposes not typically utilized in conventional synthetic biology: for example, in circumstances when a product of one genetic cascade is toxic to one or more parts of a second cascade, or in methods of tuning two genetic cascades that require dramatically different concentrations of a co-factor-there are numerous examples throughout chemistry of reactions being run under specialized, and thus necessarily isolated, reaction conditions. Certain embodiments of SMC circuits of the invention (e.g., SMC-based circuits) can operate in parallel with other SMC circuits of the invention without crosstalk between the circuits. Thus, certain aspects of the invention include populations of SMCs that respond differently to the same external activator and use of such SMCs. As noted above, the sensitivity of and selection of particular therapeutic or diagnostic agents for a specific disease is another example of the fine tuning that such compartmentalization can provide.

In some aspects, the invention includes multiple genetic circuits prepared in separate populations of SMCs, wherein communication modalities between the populations are present. In this way, compositions of the invention include entire compartmentalized genetic circuits-which allows the circuits to be separated (also referred to as being “isolated” from others) for reasons of control fidelity, toxicity, or reagent tunability- and to connect one or more compartmentalized circuits of the invention to other compartmentalized circuits. This aspect of the invention permits modularity between genetic circuits by physically separating circuit elements into different SMCs.

Although certain reactions are possible using well-compartmentalized environments of SMCs of certain aspects of the invention, some embodiments of SMCs of the invention can be used to bring together two or more genetic cascades into one environment at a particular time. For example, SMCs of the invention can be used in situations where two precursors require synthesis in different milieus, but then ultimately must be reacted with one another. As another non-limiting example, one or more proteins can be expressed at high yield in a bacterial expression system using an SMC of the invention, and the protein may receive post-translational modifications from eukaryotic cell lysate. As noted above, any of the foregoing examples can be applied to the therapeutic delivery of agents.

As used herein a synthetic minimal cell (SMC) is a liposome bioreactor that under suitable conditions is able to perform some of the biochemical functions of a living cell, in one embodiment most notably transcription and in one embodiment transcription and translation for the expression of nucleic acids or proteins. SMCs of the invention may be prepared using methods described herein in conjunction with known methods for vector preparation, gene selection, recombinant techniques, expression conditions, etc. known in the art. In one embodiment, the one or more proteins produced in a SMC may produce a therapeutically or diagnostically useful small molecule compound, from a precursor in the same SMC or produced by another SMC, that, in one embodiment, may be produced when the SMC senses a disease trigger.

An SMC of the invention may comprise one or more expression vectors, also referred to as expression constructs. As used herein, the term “vector” refers to a nucleic acid molecule capable of transporting between different genetic environments another nucleic acid to which it has been operatively linked. The term “vector” also refers to a virus or organism that is capable of transporting the nucleic acid molecule. Vectors capable of directing the expression of genes to which they are operatively linked are referred to herein as “expression vectors”.

A non-limiting example of an expression vector used in SMCs and methods of the invention may be a plasmid or virus that includes one or more elements such as a gene of interest, an enhancer, a promoter, etc. In certain aspects of the invention, a promoter may be an inducible promoter. An expression vector introduces one or more genes of interest into an SMC of the invention. Under appropriate conditions, (as a non-limiting example-when triggered or induced) the presence of expression vector results in expression of at least one polypeptide of interest in the SMC. A vector useful in methods and SMCs of the invention may include regulatory sequences such as one or more of an enhancer region and a promoter region that participate in effective transcription of a gene of interest also included in the vector.

In certain aspects of the invention, non-limiting examples of a polypeptide of interest may be: a membrane channel polypeptide (also referred to herein as a “pore”), a membrane pump polypeptide, a detectable label, an agonist polypeptide, an antagonist polypeptide, a therapeutic polypeptide, a diagnostic polypeptide, a polypeptide that triggers expression of a second polypeptide, etc. In some embodiments of the invention a channel polypeptide or a membrane pump polypeptide may be light-activated polypeptides, which are also referred to as optogenetic polypeptides. As used herein, the term “channel” refers to a membrane channel protein that permits transport of agents across a cell membrane. As used herein, the term “agent” may be used in reference to a channel, may be a small molecule, an ion, a polypeptide, etc. Crossing through a membrane channel may occur via active or passive transport. Membrane channels and agents that cross membrane channels are routinely prepared and utilized in the art and means for their preparation and use will be understood by the skilled artisan and their use is routinely practiced in the relevant arts.

Non-limiting examples of molecules that may be included in SMCs of the invention are vectors and their encoded polypeptides. Examples of encoded polypeptides that may expressed in SMCs of the invention include, but are not limited to: channel polypeptides, pore polypeptides, opsin polypeptides, detectable label polypeptides, trafficking polypeptides, signal polypeptides, export polypeptides, etc.

Non-limiting examples of detectable label polypeptides, that may be expressed in an SMC of the invention include: green fluorescent protein (GFP); enhanced green fluorescent protein (EGFP), red fluorescent protein (RFP); yellow fluorescent protein (YFP), tdTomato, mCherry, DsRed, cyan fluorescent protein (CFP); far red fluorescent proteins, luciferase, etc.

Non-limiting examples of promoters that may be included in SMCs of the invention are mammalian and bacterial promotors such as, but not limited to, Lac, T7, P70, human ubiquitin C (UBC), PBAD, promoters and functional variants thereof. Methods to select and include promoters in vectors are well known in the art.

Therapeutic agents that can be produced by SMCs of the invention can be any agent a SMC can be programmed to produce. In certain embodiments, the therapeutic agent is present within the SMC and the SMC is programmed to produce a means to release or deliver the therapeutic agent. In certain embodiments, the SMC is programmed to produce both the therapeutic agent and the means to release or deliver it.

Nonlimiting examples of therapeutic agents include nucleic acid based therapeutic agents, such as but not limited to DNA, RNA, mRNA, antisense RNA, shRNA, siRNA or CRISPR sgRNA. Non-limiting examples of therapeutically useful siRNAs include siRNA binding to any number of target transcripts of therapeutic interest. Nucleic acids may have, in one embodiment, a chemically modified sugar or nucleobase.

In one embodiment the therapeutic agent is a protein. In one embodiment, the protein is a part of an antibody molecule, or a fragment of an antibody, or an antigen-binding fragment of an antibody.

Nonlimiting examples of therapeutic agents include protein based therapeutic agents including antibodies and other antigen-binding polypeptides, enzymes, toxic enzymes, antibiotics, antimicrobial peptides, prodrugs, cytokines, and chemokines.

In one embodiment the therapeutic agent is a peptide such as the hormones insulin, ACTH, calcitonin, oxytocin, vasopressin, octreotide, somatostatin and leuprorelin; and the anticancer peptides hepcidin, dermaseptin, PTP7, MGA2, HNP-1, temporin, and NK-2. These are merely exemplary and nonlimiting as to the selection of peptide that can be output by a SMC of the invention.

In one embodiment the therapeutic agent may be a nucleic acid enzyme (ribozyme), a catalytic peptide, a membrane-disrupting protein, a transporter protein, a signal protein, a chemokine or a cytokine.

In one embodiment, the therapeutic agent is a therapeutically useful enzyme such as tissue plasminogen activator, streptokinase, lysozyme, urokinase, plasmin, bromelain, trypsin, collagenase, lactase, glucocerebrosidase, alglucerase, asparaginase, imiglucerase, tenectaplase, beta-lactamase and hyaluronidase. These are merely exemplary and nonlimiting as to the selection of protein that can be output by a SMC of the invention. Other non-limiting examples of therapeutic proteins and diseases they are used to treat include human insulin for diabetes, erythropoietin for anemia and chronic renal failure, interferon-beta and gamma for cancer, DNase for pulmonary treatment, vaccines for hepatitis B, interleukin-2 for AIDS, and many different kinds of monoclonal antibodies for diagnosis and treatment of, for example, breast and lung cancers. In one embodiment, targeted delivery of any one of these therapeutic agents at a particular site in the body or in response to the presence of a particular signal in the body provides more specific local efficacy and reduced systemic effects than parenteral or other delivery of the agent.

In one embodiment the therapeutic agent is an antibiotic or antimicrobial peptide such as bleomycin, actinomycin, vancomycin, dermicidin, cecropin, moricin, melittin, magainin, abaecin, indolicin, protegrin, tachyplesin. These are merely exemplary and nonlimiting as to the selection of antibiotics and antimicrobial peptides that can be output by a SMC of the invention.

In one embodiment the therapeutic agent is an antibody, antibody fragment, or an antigen-binding fragment of an antibody or a related molecule. In one embodiment the antigen-binding agent is a scFV, Fab or F(ab′)2. These are merely exemplary and nonlimiting as to the selection of antigen-binding agents that can be output by a SMC of the invention.

In one embodiment the therapeutic agent is a small molecule that is a product of a biosynthetic pathway involving at least one protein, such as an enzyme. Non-limiting examples of therapeutically useful small molecules that can be produced in a SMC include artemisinin, beta-carotene, nitrous oxide, lycopene, natamycin, plicamycin, novobiocin, dactinomycin, doxorubicin, tyrothricin, daptomycin, tetracycline, bacitracin, ampicillin, kanamycin, trimethoprim, spectinomycin, streptomycin, chloramphenicol, piperacillin, viomycin, capreomycin, gentamicin, erythromycin, neomycin, vancomycin, ristocetin, daunorubicin, streptozocin, fusidic acid, rifampicin, bleomycin, mitomycin, aztreonam, mupirocin, cycloserine, tobramycin, sirolimus, pristinamycin, virginiamycin, elsamitrucin, toyocamycin, friulimicin, sparsomycin, indirubin, enviomycin, alanosine, monensin, and bafilomycin.

In one embodiment, the therapeutic agent is present in the SMC as a pro-drug or precursor, and genetic circuit produces the polypeptide, e.g., an enzyme, that activates the pro-drug into the therapeutic agent. In one embodiment the pro-drug is an antimicrobial agent prodrug.

In one embodiment the therapeutic agent is an RNA silencing agent such as an antisense RNA, siRNA, shRNA, and anti-micro RNA. In one embodiment, the agent is patisiran and givosiran. These are merely exemplary and nonlimiting as to the selection of therapeutic RNAs that can be output by a SMC of the invention.

Nonlimiting examples of therapeutic agents include small molecules such as drugs, toxins, antibiotics and prodrugs. In one embodiment, the small molecule is a product of a genetic circuit as described above, which converts at least one precursor (or intermediate) of the small molecule to the therapeutic small molecule upon activation of the SMC. The biosynthetic pathway may comprise multiple steps. The biosynthetic pathway may use precursors present in the SMC or from the exterior or the SMC, such as in a patient's circulation or a metabolite or other compound at or near the site of disease. Examples of such small molecules and biosynthetic pathways comprising one or more steps include artemisinin, beta-carotene, nitrous oxide and lycopene. In one embodiment, the precursor or intermediate contained within the SMC is not permeable to the SMC membrane, but the membrane is permeable to the therapeutic small molecule.

Any of the foregoing examples pertaining to a therapeutic agent are equally applicable to a diagnostic agent, such as a reporter or marker, that is released by the SMC and detectable in, for example, a bodily fluid of the patient, the agent then informing the diagnosis or informing optimal or appropriate treatment. Thus, diagnostic agents that can be produced by SMCs of the invention can be any agent a SMC can be programmed to produce. In certain embodiments, the diagnostic agent is present within the SMC and the SMC is programmed to produce a means to release or deliver the diagnostic agent. In certain embodiments, the SMC is programmed to produce both the diagnostic agent and the means to release or deliver it. Any description herein regarding therapeutic agents are applicable to diagnostic agents as well.

Methods for selecting and using trafficking sequences, signal sequences, export sequences, promoters, etc. in vectors for expression as fusion proteins are known in the art, see for example: Chow, X. et al., Nature 463, 98-102 (2010), Gradinaru, V. et al., Brain Cell Biol. 36, 129-139 (2009); and Kugler, S. et al., Gene Therapy 10, 337-347, (2003). The content of each of the above references is incorporated herein by reference in its entirety. Those skilled in the art will be able to use routine methods to prepare vectors encoding trafficking, signal sequences, export sequences, etc. for use in certain embodiments of SMCs of the invention.

Expression vectors and methods of their use for expression of numerous different types of polypeptides are well known in the art. Non-limiting examples of suitable expression vectors and methods for their use are provided herein. A skilled artisan will understand how to design and use expression vectors in methods and SMCs of the invention using routine procedures in conjunction with the disclosure provided herein.

As used herein, the term “plurality” used in reference to SMCs of the invention, means: at least 2, 3, 4, 5, 6, 7, 8, 9, 10, 50, 100, 1000, 10,000 or more SMCs. In some aspects of the invention, each SMC in a plurality of SMCs includes the same one or more: expression vectors, genes of interest, and may be induced to express one or more genes by the same agents as the other SMCs in the plurality of SMCs. In certain aspects of the invention each SMC in a plurality of SMCs may include one or more different: expression vectors, genes of interest, internal environment than one or more other SMCs in the plurality and may be induced to express one or more genes by at least one different agent than induces expression of a gene of interest in one or more of the other SMCs in the plurality of SMCs. Thus, in some aspects of the invention a plurality of SMCs may be homogeneous and in certain aspects of the invention a plurality of SMCs may be heterogeneous.

In certain aspects of the invention, an SMC of the invention, can be programmed to be fused together with another SMC. SMC fusion can be implemented using any suitable fusion system, including but not limited to a system utilizing SNARE/coiled-coil hybrid proteins, which can be generated in complementary pairs that are specific in their fusion properties [see for example: Meyenberg, K. et al., Chem. Commun. 47, 9405 (2011) and Robson Marsden, H. et al., Biomater. Sci. 1, 1046 (2013), each of which is incorporated by reference herein in its entirety]. In the non-limiting SNARE fusion system example, complementary fusion elements can be packaged into separate populations of SNARE-fusable SMCs of the invention and the SMCs can be fused together. In certain embodiments of the invention, fusion elements are present on the exterior surface of an MSC. In some aspects of the invention, complementary fusion elements are present on the exterior surfaces of two or more SMCs in a population of SMCs. The fraction of occupied SMC within each population can be independently calibrated making it possible to tune the overall production of the final output, as well as the degree of modulation by environment. In some aspects of the invention, SMCs undergoing SNARE-mediated fusion may form large aggregates made from multiple starter SMCs. In one embodiment, fusion of particular SMCs to produce a therapeutic agent or diagnostic agent useful for the treatment or diagnosis of a certain disease may be provided.

### Uses of SMCs of the Invention

As described herein, the SMCs of the invention are useful for the delivery of therapeutic agents to a patient in need of treatment and more particularly to a site within the body that optimizes the therapeutic efficiency of the delivered agent or agents. As noted herein, the sensor, production and outputting features of the SMCs are designed to (1) identify when and where the therapeutic agent is needed; which information is conveyed to (2) production of the therapeutic agent and then (3) release of the therapeutic agent to the bodily site to exert its beneficial effect on the disease or condition intended to be treated. Also as noted, one or more SMCs may participate in this process, such that one or more sensor SMCs may signal one or more production SMCs to produce the therapeutic agent or biosynthetic intermediates that may then be converted in one or more steps to the desired therapeutic agent by the same or different SMCs, and outputted at the desired site. Thus, in one embodiment, the aforementioned three steps may reside in a single SMC or be distributed among multiple SMCs. Thus, in one embodiment, “a SMC” may comprise multiple SMCs that together serve the purpose of the invention. Furthermore, SMCs of the invention are also useful for the diagnosis of disease by releasing an agent detectable at the site or in a bodily fluid of the patient, identifying the disease, for example, the type of infection, informing appropriate treatment.

In one embodiment, the SMC of the invention can serve to enhance or complete the production of an endogenous biologically active compound where the source of such compound in the body is lacking or insufficient. In one non-limited example, production of a hormone or neurotransmitter required for a biological process but deficient in a subject may be enhanced by administration of SMCs that detect the site or cell types normally responsible for producing the compound, and producing it at the site. In another embodiment, a SMC can sense the increased or decreased levels of a circulating or local compound and in response produce an agent that decreases or increases, respectively, the levels or biological activity of the compound.

Before further describing the potential scope of uses of the invention, a description of specific examples of SMCs to achieve the aforementioned purposes is warranted. These examples are merely exemplary and are not intended to be in any way limiting the scope of the invention. One of skill in the art can design other SMCs systems that deliver other therapeutic agents and sense conditions where release of such therapeutic agents are desired, adhering to the scope and intent of the invention.

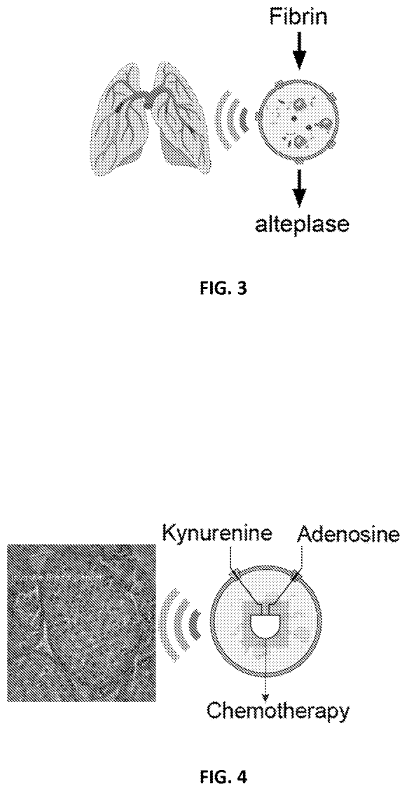

In one non-limiting example, use of SMCs to treat a blood clot in a blood vessel is used (FIG. 3), wherein the therapeutic agent is a thrombolytic agent such as the proteins (enzymes) tissue plasminogen activator (tPA) or streptokinase. These enzymes activate human plasmin, which breaks down the fibrin in clots and clears the embolism. However, systemic or even local administration of thrombolytics is often contraindicated by conditions such as recent surgery, and other interventions such as thrombectomy can be risky. By using SMCs to target clots and generate tPA only in response to the presence of clot biomarkers, the current therapeutic index of thrombolytic drugs can be markedly improved. In one embodiment the sensor of the SMC detects the presence of fibrin, a component of a blood clot. This can be achieved using a fibrin-binding antibody or aptamer on the surface of the SMC as a targeting moiety. In another embodiment the sensor detects increased concentrations of heme. In another embodiment the sensor detects low oxygen. Multiple sensors may be used to assure that the therapeutic agent is produced only at a site where multiple triggers are located. The targeting moiety that senses these conditions may be conjugated to the outside of the SMC directly or to other surface-conjugated molecules. The sensor upon detecting a clot activates the production of the thrombolytic enzyme from a genetic circuit containing the elements to express the enzyme from a DNA-containing construct within the SMC. In addition to activating enzyme production, the genetic circuit also generates a transporter for the therapeutic enzyme to output from the SMC. In addition, in one embodiment, the SMCs are engineered to have a specific operating lifetime such that continue outputting of the thrombolytic is stopped before side effects can arise, or the sensor upon detecting the lack of the clot or conditions characterizing a clot, stop the genetic circuit and stop production of the enzyme. In one embodiment, the genetic circuit includes a timer that after a certain time period shuts down the functional operation of the SMC.

In another example where the therapeutic agent is a small molecule, the small molecule drug is catechol (ortho-benzenediol). A SMC of the invention comprises catechol diethyl ether (1,2-diethoxybenzene) and the genetic circuit includes DNA coding for the enzyme that hydrolyzes aromatic ether bonds, and RNA polymerase and cell-free protein translation system. Upon external stimuli at specific tissue, the SMC expresses the enzyme, the enzyme hydrolyzes ether bonds in 1,2-diethoxybenzene, producing catechol. Catechol is SMC membrane permeable so that it is exported without the need for an active transport mechanism or a membrane pore.

In another embodiment, SMCs of the invention act in cooperation as a synthetic immune system. Certain SMCs detect the presence of cancer biomarkers such as adenosine or kynurenine and in response produce and release cytotoxic proteins and small molecules. Other SMCs produce and release chemoattractant molecules such as chemokines such as IL-8 or CLL5 that recruit other immune cells to the site of the malignancy.

In another example, a consortium of SMCs is used as a “broad spectrum antibiotic”. In this embodiment, the consortium comprises multiple types of SMCs (members) in which each member type can detect a specific species or type of pathogen. SMCs are programmed with membrane-bound or soluble sensors that specifically detect molecules specific to each species or type of pathogen, or an SMC member can be comprise a membrane that lyses upon interaction with a specific bacterial agent, such as a pore protein (e.g. alpha-hemolysin) or other agent. Sensor molecules (proteins, RNA, DNA, etc.) detect molecules characteristic of a specific pathogen (metabolites, quorum sensing molecules, toxins, membrane components, etc.) which then activates the appropriate circuit to either produce an enzyme to then produce the appropriate antibiotic (FIG. 1B) and/or a pore to release an encapsulated antibiotic. The membrane of the liposome itself can be composed such that it is susceptible to lysis by specific bacterial toxins as well. Thus, multiple populations of types of SMCs (members) can be simultaneously co-administered as a consortium to provide simultaneous protection against multiple classes of pathogens.

In another embodiment, in addition to producing antibiotics and pore proteins as described above, the SMCs can also be engineered to produce reporters (proteins, dyes, DNA, RNA, small molecules, etc.) that can then be detected in urine or blood and used to aid in the diagnosis of infections. A consortium of a population of SMCs each reporting a different species or type of bacterium would provide a diagnosis of the type of infection. Currently, the treatment of bacterial infections is limited by the fact that assays to determine the class of pathogen take 24-48 hours. Knowledge of the class of pathogen a patient is infected with is critical to determining which type of antibiotic to administer. Given that bacterial infections can progress rapidly, the current standard of care is to begin treatment immediately broad-spectrum antibiotics, before the nature of the pathogen is known. While these antibiotics can be effective, they can have significant toxicities and their overuse contributes to the development of antibiotic-resistant bacteria.

Thus, by injecting such a consortium of bacterial species- or type-recognizing SMCs into a patient with a bacterial infection, member SMCs will recognize specific pathogens and only release species or type specific antibiotic if these pathogens are detected, or release reporters of that bacterial species or type that can be detected in a bodily fluid of the patient enabling the patient to be treated immediately with the assurance that they will only receive the appropriate drug.

In one embodiment, SMCs or liposomes of the invention may comprise antibiotics that are released only upon sensing by the SMC the presence of the appropriate bacterial species or type for that antibiotic. In one embodiment, the antibiotic is present in the SMC. In one embodiment, the sensor activates the genetic circuit to produce the antibiotic.

Sensors. In other examples, a sensor module that will detect relevant disease biomarkers includes, for tumors, cancer surface markers such as CD19, and release of metabolites such as kynurenine and adenosine, tumor microenvironment markers such as low oxygen or low pH, (FIG. 4) which alone or together may indicate the presence of a tumor. In the example of bacterial infection, the SMC will detect bacterial cell surface molecules or bacterial species-specific soluble molecules (and as noted herein, a consortium of SMCs may be provided comprising individual types of SMCs each of which can detect a different bacterial species). The sensor module may be composed of a membrane-anchored receptor module or a free-floating molecule inside the SMC. The sensor module can either be chemically altered by the sensed chemical or the sensed chemical can interact with the sensor in such a way as to alter its conformation and change its activity. The sensor can either modify gene expression directly or it can interact with a transducing molecule that will then alter gene expression. Gene expression is modified when the sensor or transducing molecule binds near the promoter of the target gene.

Binding of the sensor or transducer alters the transcription rate from the promoter, which changes the levels of gene expression and subsequent target protein. The term activator may be used herein to refer to the stimulus for the sensor.

In some embodiments, a stimulus for activating the genetic circuit of a SMC may require a second or additional stimulus in order to prevent activation except when all conditions are met at the site where the therapeutic agent is desired. This failsafe mechanism limits non-specific interactions such as for using SMCs essentially to perform microsurgery of, for example, a tiny tumor in the brain, where a therapeutic (chemotherapeutic) agent would have significant side effects if released in an undesired location. As noted above, two or more biomarkers for a tumor may be required for activating the genetic circuit, such as one surface marker and one metabolite; one surface marker and one microenvironmental marker; one metabolite and one microenvironmental marker; or one surface marker, one metabolite and one microenvironmental marker.

In one embodiment, the stimulus could be internal (e.g., presence of cancer metabolite at the site of solid tumor, or presence of an inflammatory marker on the site of injury), or in one embodiment, external (e.g., irradiation of a site with light, electrical stimulation, or delivery of additional small molecule to activate the production of drug inside SMC). In one embodiment the stimulus may activate the genetic circuits in a subpopulation of SMCs among a consortium, the subpopulation having a sensor responsive to the particular stimulus. Thus, a consortium of SMCs can be used to treat or diagnose a disease wherein the pathological agent is not known or could be among multiple possibilities.

The stimulus can also be the deployment of molecules from another SMC or therapeutic device.

Genetic circuits. A genetic circuit of a SMC of the invention is a circuit that will compute to what degree each relevant biomarker signal is present and correspondingly titrates the amount of output therapeutic or diagnostic nucleic acid or therapeutic or diagnostic protein, or proteins (enzymes) that participate in a biosynthetic pathway within the SMC to produce a therapeutically or diagnostically useful small drug (or, in other embodiments, converting an inactive protein into an activated protein, or producing a pore or transporter molecule to allow release from the SMC of an agent within or produced within the SMC). The circuit is composed of a system of genes for proteins that can further regulate gene expression. The organization of the regulatory structure for these genes dictates the information flow through the system. When the system is organized in certain ways, it can perform computations, either digital or analog. Sensor modules are integrated “upstream” of the circuits and feed inputs into the circuits. Modules for producing therapeutic molecules are integrated “downstream” of the circuits and are completely controlled by the activity of the circuit. Thus, production of therapeutic or diagnostic protein can be carefully controlled in a way that is conditional to the input signals. Circuit controls also allow for the careful titration of output molecule, enabling the SMC to dispense output molecules as needed. The circuit may also include a timer to stop the production of the therapeutic or diagnostic protein (or other agent) after a certain temporal duration, or another sensor that can detect a positive therapeutic effect of the therapeutic agent and upon sensing the positive therapeutic effect, initiate a circuit to stop production, or produce a diagnostic protein or other agent, to indicate therapeutic success.

Output. As noted above, the therapeutic agent may be permeable to the SMC membrane and diffuse into the desired location, or the SMC may contain membrane pore protein or other means for outputting the therapeutic or diagnostic agent. These represent passive output or release mechanisms. In one embodiment, the output is a controlled release. In one embodiment, the genetic circuit produces a molecule that enables release of the therapeutic or diagnostic agent from the SMC such as a transporter protein or holin protein. This module is controlled by the circuit and generates output only when certain conditions are met. The module may also contain enzymes or chaperones required to activate or modify the therapeutic or diagnostic molecule or facilitate its transport across the liposomal membrane. Controlled release may also involve either second type of external stimulus or the SMCs could be permeabilized by light or chemical lipid transformation.

### EXAMPLES

FIG. 1 depicts the overall concept of the invention using SMCs as programmable therapeutic agents. The SMCs can comprise a sensing function, such as having an alpha-hemolysin pore to permit entry of small molecules which subsequently activates the circuit; one or more circuits that responds to the sensing function, where the presence or one or more signals activates one or more genetic circuits; and outputs, for example, a small molecule or protein present in the SMC such as the opioid receptor mu (OPRM1), adenylate cyclase IIC2 (AC-IIC2), adenylate cyclase 3 (AC3) or green fluorescent protein (GFP). Each has therapeutic or diagnostic value when released from SMCs in response to a particular signal detected by the sensor. As described above, the genetic circuit activated by the sensing can produce the therapeutic or diagnostic agent through cell-free transcription/translation components present in the SMC; in another embodiment, the MC contains the therapeutic or diagnostic agent, and the genetic circuit, activated by the sensing, can produce a pore or other product that releases the therapeutic or diagnostic agent contained therein; wherein the agent is released through the pore, or rupture of the SMC membrane.

FIG. 2 depicts some of the potential uses of the SMCs of the invention for sensing of and responding to diseased cells, tissues or other pathologies, by producing and releasing, or releasing, a therapeutic or diagnostic agent such as but not limited to: detection of beta-amyloid in the brain or other locations and producing a signal that is diagnostic for Alzheimer's disease; sensing of 2-hydroxyglutarate, a metabolite associated with liver cancer, where the SMC then produces and releases, or releases, an anticancer agent; sensing of glypican-1, a cancer-associated glycoprotein, and producing a diagnostic signal for pancreatic cancer; sensing of N-terminal pro-B-type natriuretic peptide (NT-proNBP), and producing a signal that is diagnostic for heart failure; and sensing of osteopontin production, a colon-cancer associated signaling protein and marker that results in the SMC producing and releasing, or releasing, an anti-osteopontin antibody to block local signaling. These examples are merely exemplary of the various uses of the SMCs of the invention, and are not intended to be limiting.

### Example 1

### SMC that Delivers tPA at the Site of a Blood Clot

As depicted in FIG. 3, SMCs can be made to treat a blood clot (embolism), wherein the SMCs comprise a sensor for fibrin, a genetic circuit to produce tissue plasminogen activator (tPA; alteplase) and a membrane-disrupting protein, channel protein, or secretion mechanism to output the tPA from the SMC to the clot. tPA activates human plasmin, which breaks down the fibrin in clots and clears the embolism.

A fibrin-binding antibody or aptamer is provided on the surface of the SMC as a targeting moiety. The sensor upon detecting a clot activates the production of the thrombolytic enzyme from a genetic circuit containing the elements to express the enzyme from a DNA-containing construct within the SMC: a promoter, and a tPA CDS, and the subcellular components to facilitate transcription and translation. Also included in the genetic circuit is a membrane-disrupting protein that breaks the SMC membrane. The genetic circuit is designed such that the SMC comprises a therapeutically active amount of tPA before sufficient membrane-disrupting protein is produced to release the tPA in the proximity of the clot. Thus the release of the enzyme is in proximity of the clot, increasing efficacy, and potential adverse systemic effects are obviated.

### Example 2

### SMC that Delivers Catechol

SMCs are made that contain the catechol precursor catechol diethyl ether (1,2-diethoxybenzene) and DNA coding the enzyme that hydrolyzes aromatic ether bonds, plus RNA polymerase and cell-free protein translation system. Upon external stimuli at a specific tissue, the SMC expresses the enzyme, the enzyme hydrolyzes ether bonds in 1,2-diethoxybenzene producing catechol which diffuses out of the SMC to the site.

### Example 3

### SMC that Delivers a Chemotherapeutic Agent at the Site of a Tumor

SMCs to treat a cancer can comprise a sensor for tumor-specific metabolites, markers, or environmental conditions; a genetic circuit to compile the various input signals, and a module to produce one or more cytotoxic or other proteins that facilitate killing of tumor cells and produce a protein that releases the cytotoxic or other proteins. In another embodiment, the SMC is prefilled with an anti-cancer agent, and a module produces a protein that releases the anti-cancer agent from the SMC.

A tumor-binding antibody or aptamer is provided on the surface of the SMC as a targeting moiety sensor. The sensors upon detecting a tumor cell or tumor environment activate the production of the therapeutic and membrane disrupting proteins (or membrane disrupting protein in the case of a SMC prefilled with an anticancer agent) via a genetic circuit containing the elements to express the therapeutic and/or membrane-disrupting protein from a DNA-containing construct within the SMC: a promoter, and a protein CDS, and the subcellular components to facilitate transcription and translation. As shown in FIG. 4, a SMC that has sensors for both kynurenine and adenosine, which together activate the production and/or release of a chemotherapeutic agent by the SMC at the site of detection of these metabolites. The presence of kynurenine and adenosine together is, in one embodiment, indicative of invasive breast cancer. This is a non-limiting example of SMCs that can detect and respond to a particular signal and release a therapeutically effective agent. In this example, the presence of the two signals is required to release the agent.

### Example 4

### Consortium of SMCs that Identifies a Bacterial Infection and Counteracts It

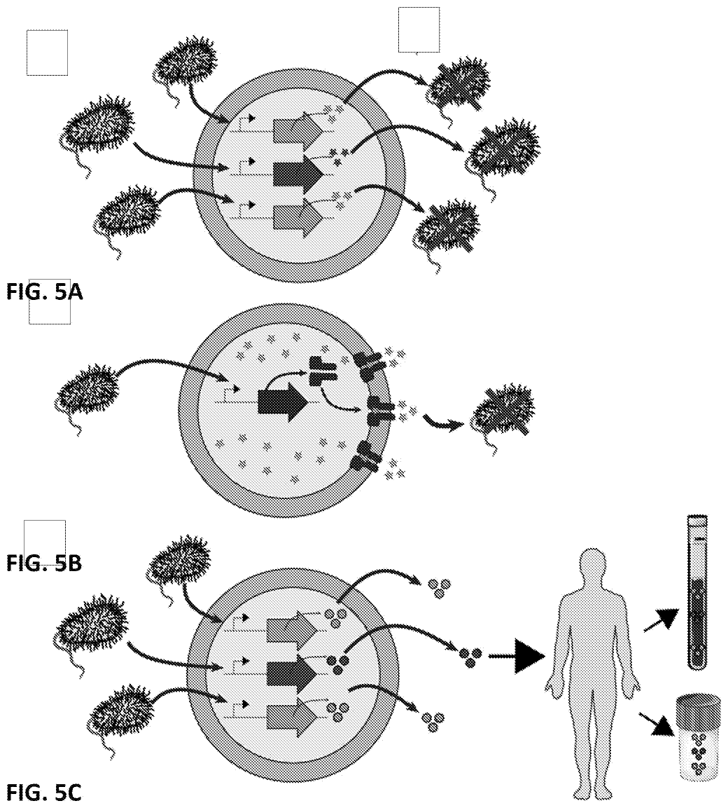

As shown in FIG. 5, SMCs can be made that sense a particular bacterial species or bacterial type which activate either (A) the production of an antibiotic or antimicrobial agent specific for that bacterial species or type; (B) the production of a pore or other membrane-lysing agent that allows release of a bacterial species- or type-specific antibiotic or antimicrobial agent present in the SMC; or (C) the production of a reporter or detectable marker of that bacterial species or type, the reporter or detectable marker which than can be identified in a bodily fluid sample from the subject, and indicate the appropriate therapeutic regimen to treat that bacterial species or type. In one embodiment, the marker or reporter, other than identifying the bacterial species or type, does not have to have any particular relationship with the bacterium. By way of non-limiting example, the reporter may be an nuclease-resistant oligonucleotide or a retro-inverso peptide that can be detected at high sensitivity in a blood sample. Different oligonucleotides or peptides are associated with SMCs that detect different pathogens. Analysis of a blood sample, such as by LC-MS/MS or ELISA, from before and after administration of the SMCs can identify which particular oligonucleotide or peptide appeared in the sample, and thus the nature of the pathogen.

A consortium of SMCs can comprise of more than one type of such SMCs, each of which is specifically programmed to detect a specific species of bacteria and respond by releasing or generating an antibiotic (e.g., SMC capable of responding to one species shown in FIG. 5A or 5B).

As shown in FIG. 5C, a doctor may apply a medicinal formulation of this consortium to the site of a patient's suspected infection. If a bacterial infection is present, the bacteria will produce small molecules such as quorum sensing molecules, waste products, peptides, oligonucleotides, or proteins uniquely associated with that species, which can be detected from a sample from the site or from circulation. In another embodiment, one type of SMC in the consortium will sense the presence of those one or more unique molecules through its sensor module. The responding circuit module will then generate an enzyme that can turn a colorless substrate into a colored biomarker (diagnostic) and/or a pathway of enzymes that generates an antibiotic molecule and/or a pore protein that releases co-encapsulated antibiotic molecule (therapeutic). The response of each type of SMC will be proportional to the amount of infection. This enables the SMC to titrate the amount of antibiotic released to a level appropriate for the scale of the infection. This avoids overdosing the patient or causing unneeded production of antibiotic.

### Example 5

### Light-Sensitive SMCs that Release a Chemotherapeutic Agent

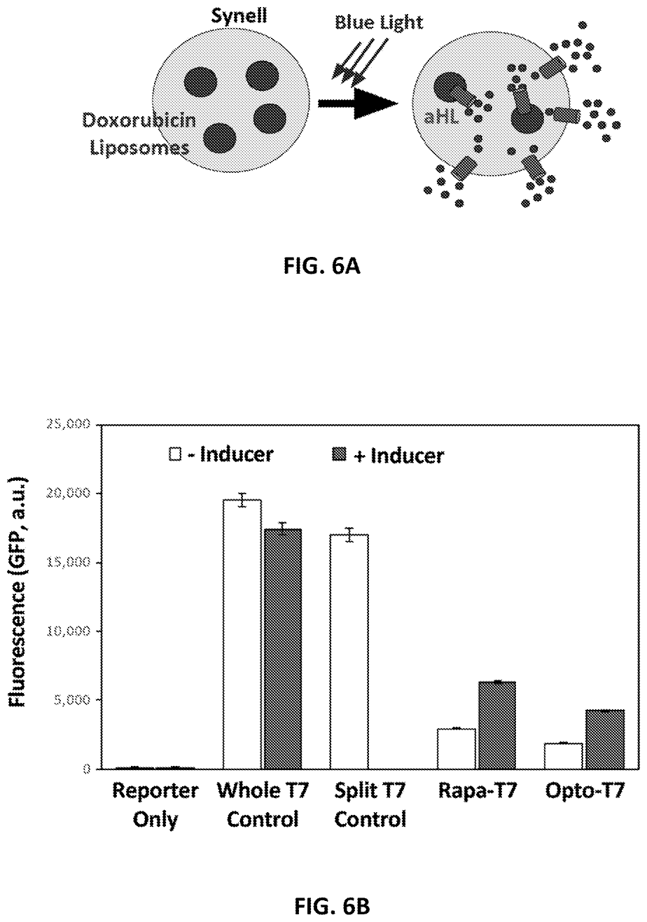

Another example of a SMC that can sense and respond for therapeutic use is shown in FIG. 6. FIG. 6A depicts a scheme by which SMCs comprising nested liposomes containing the chemotherapeutic doxorubicin and circuitry expressing blue light-sensitive split T7 RNA polymerase. The T7 promoter in this system expresses an alpha-haemolysin (aHL) pore complex. Upon exposure of the SMCs to blue light, the blue light-sensitive split T7 RNA polymerase is activated which then activates the production of aHL from the genetic circuit. The aHL forms a membrane pore that allows the doxorubicin within the SMC to be released at the site of the original activation by blue light. The induction system here can be generalized to a number of other inducible promoter systems that respond to light or small molecules such as PgnlAP2 (responds to acid production near a tumor), PhlF (responds to small molecule 2,4-diacetylphloroglucinol produced by Pseudomonas), luxR (responds to quorum sensing molecules such as acyl-homoserine lactones that are produced at the site of infection). A split T7 system that responds to the drug rapamycin has also been produced. An integrated SMC system here comprises sensors genes, sensor promoters, and chemotherapeutic outputs encoded on a single plasmid that is encapsulated in a SMC. In the plasmid construction, the sensor proteins are encoded downstream of an unmodified, wild-type T7 promoter. Co-encapsulated wild-type T7 RNA polymerase expresses these sensor genes, essentially “priming” the SMC for its sensing task. The sensor proteins (e.g. PhlF, split T7) then assess the environment of the SMC and report either positively or negative presence of the state of interest (e.g. presence of small molecule or blue light) by either inducing or suppressing transcription of the output genes. The output genes, in this example aHL, then carry out the intended function (e.g. inducing lysis of nearby cells) either directly or indirectly via the release of other factors as in this example.

FIG. 6B shows the results of an experiment demonstrating that split T7 systems can function in cell-free systems, including a rapamycin-sensitive system (Rapa-T7) and a blue light-sensitive system (Opto-T7). In this experiment, the output T7 promoter generates a GFP protein the fluorescence of which can be measured. The unsplit T7 (“Whole T7 Control) and Split T7 system without sensing domains (”Split T7 Control) are shown as controls. Exposure of blue light to this system results in the production of the GFP similar to that in the rapamycin-sensitive T7 system. Such SMCs can be used therapeutically to be activated in situ to produce the chemotherapeutic agent locally at the site of a tumor and not systemically, which has side effects such as cardiotoxicity. High concentrations in the proximity of the tumor can be highly efficacious without systemic side effects.

### Example 6

### SMCs that Generate Pigment in situ for use in Heat Ablation

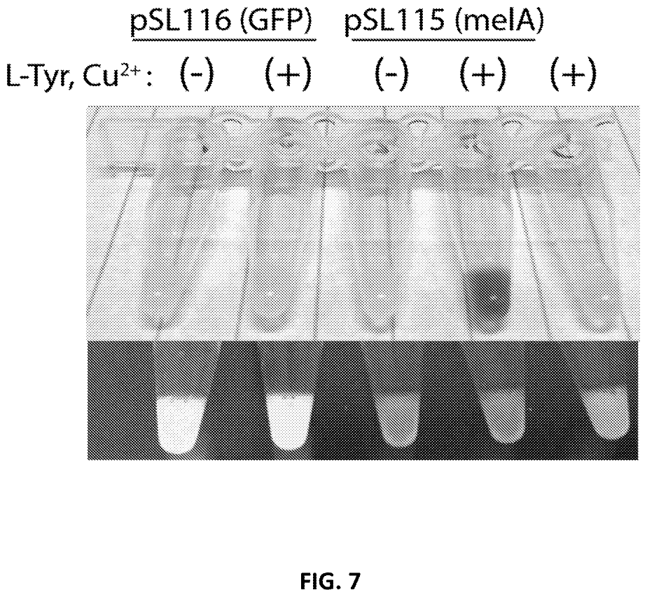

SMCs can be prepared that respond to a signal to generate a dye or pigment such as melanin, which accumulation at a site in the body can be used for subsequent heat ablation therapy. FIG. 7 shows expression of GFP and a tyrosinase (melA) in a cell-free system. Under white light (top row), a brown pigment (melanin) is produced when the melA gene, L-tyrosine, and copper ions are present. Under blue light (bottom), GFP production is observed as a control. Melanin can absorb broad-spectrum light and generate heat as a result. Such a system could be useful in using SMCs to conduct heat ablation of target tissue, comprising a sensor for a malignancy treatable by heat ablation therapy, a genetic circuit to produce melA and the molecules to produce melanin in the presence thereof. After production of brown pigment at the tumor site, heat ablation therapy can be applied to utilize the presence of the melanin to convert applied light to heat to kill tumor cells or other cells or tissues for which ablation is desired.

### Example 7

### Cytotoxicity of aHL Producing SMCs

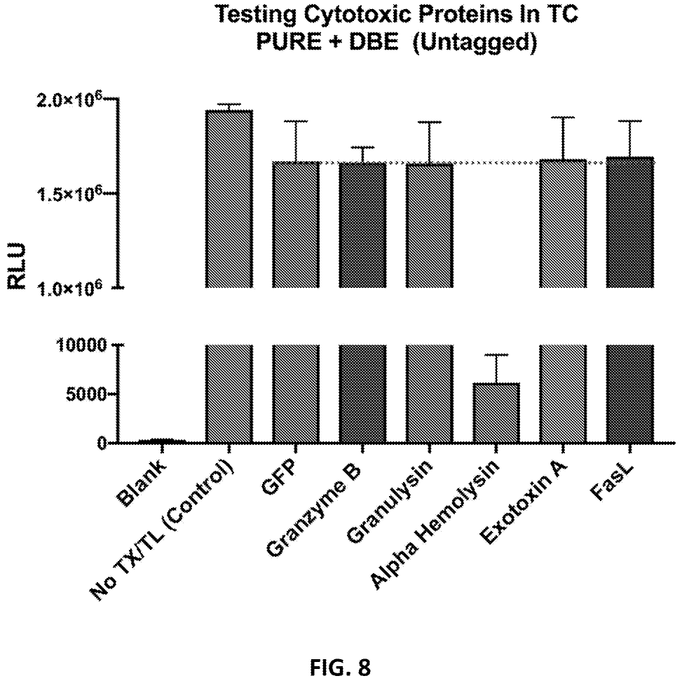

FIG. 8 shows results of a viability assay when cell-free systems generating different cytotoxic proteins are added to tissue cultures of HEK293 cells. Here, the PURE cell-free transcription-translation system containing disulfide bond enhancer (DBE) is used in order to express then correctly fold proteins with multiple disulfide bonds. When SMCs expressing alpha-haemolysin (aHL) are added to the culture, most cells are killed. No toxicity is observed from any of the other proteins. This demonstrates how SMCs could generate different proteins with specific toxic effects.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A synthetic minimal cell (SMC) or a consortium of SMCs for the production and delivery of a therapeutic or diagnostic agent, wherein each SMC comprises:
(a) at least one sensor that detects at least one condition under which the therapeutic or diagnostic agent is to be produced and delivered;
(b) at least one genetic circuit that controls the production of the therapeutic or diagnostic agent upon detection of the at least one condition by the sensor; and
(c) at least one outputting means for delivering the therapeutic or diagnostic agent outside of the SMC.

2. A synthetic minimal cell (SMC) or a consortium of SMCs for the delivery of a therapeutic or diagnostic agent, wherein each SMC comprises:
(a) a therapeutic or diagnostic agent;
(b) at least one sensor that detects at least one condition under which the therapeutic or diagnostic agent is to be delivered; and
(b) at least one genetic circuit that controls the production of an outputting agent for delivering the therapeutic or diagnostic agent outside the SMC upon detection of the at least one condition by the sensor.

3. The SMC of claim 1 or 2 wherein the therapeutic, diagnostic or outputting agent is a protein, peptide, nucleic acid or small molecule.

4. The SMC of claim 3 wherein the nucleic acid is DNA, RNA, shRNA, siRNA, CRISPR sgRNA or an antisense oligonucleotide.

5. The SMC of claim 3 wherein the agent is a protein or nucleic acid enzyme, antibody, antibody fragment, catalytic peptide, antibiotic, antimicrobial peptide, membrane-disrupting protein, transporter, signal protein, cytokine or chemokine.

6. The SMC of claim 1 or 2 wherein the therapeutic or diagnostic agent is not produced or delivered until the genetic circuit activates production of the therapeutic agent, diagnostic or outputting agent or a precursor thereof within the SMC.

7. The SMC of claim 1 or 2 wherein the sensor is membrane-bound, is soluble within the SMC, or comprises a membrane sensitive to the detected condition.

8. The SMC of claim 1 or 2 wherein the sensor detects one or more conditions that exist at a site where delivery of the therapeutic or diagnostic agent is desired.

9. The SMC of claim 8 wherein the conditions exist at the site is a result of the disease to which the therapeutic agent is desirously delivered.

10. The SMC of claim 8 wherein the one or more conditions are created at the site where the therapeutic agent is beneficially delivered.

11. The SMC of claim 10 wherein the condition is selected from radiation, heat, pH change, or the administration of an agent that targets the site desirous of beneficial therapeutic agent release and activates the SMC sensor, production, outputting or any combination thereof.

12. The SMC of claim 1 or 2 wherein the production comprises translation.

13. The SMC of claim 1 or 2 wherein the production comprises gene expression.

14. The SMC of claim 1 or 2 wherein the production is controlled by transcription factors.

15. The SMC of claim 1 wherein the outputting means is by passive or controlled release from the SMC.

16. The SMC of claim 1 wherein the outputting means is responsive to a condition different from that of the sensor.

17. The SMC of claim 1 or 2 further comprising a negative feedback sensor that reduces production or outputting of the therapeutic agent.

18. The SMC of claim 1 or 2 wherein the sensor means, production means and outputting means of the therapeutic or diagnostic agent occurs in one, or two or three different SMCs.

19. The SMC of claim 2 wherein the outputting agent is a protein, a peptide, a small molecule, a membrane channel polypeptide (also referred to herein as a “pore”), a membrane pump polypeptide, a trafficking polypeptide, a signal polypeptide or an export polypeptide.

20. The SMC of claim 1 or 2 comprising at least a portion of at least one multi-gene genetic circuit.

21. The SMC of claim 1 or 2, wherein the SMC comprises at least one multi-gene genetic circuit.

22. The SMC of claim 1 or 2, wherein the SMC comprises a portion of a multi-gene genetic circuit and a second SMC includes a second portion of the multi-gene genetic circuit.

23. The SMC of claim 1 or 2, wherein the multi-gene genetic circuit comprises 2, 3, 4, or more gene components.

24. The SMC of claim 1 or 2, wherein the portion of the multi-gene genetic circuit comprises 1, 2, 3, 4 or more gene components.

25. The SMC of claim 23 or 24, wherein contacting the SMC with an activator modulates an activity of at least one gene component of the genetic circuit.

26. The SMC of any one of claim 23 or 24, wherein an activity of a first gene component of the SMC modulates an activity of one or more additional gene components of at least one of: (1) the multi-gene genetic circuit of the SMC and (2) a multi-gene genetic circuit of another SMC.

27. The SMC of claim 26, wherein the multi-gene genetic circuit of (2) is different than the multi-gene genetic circuit of (1).

28. The SMC of any one of claims 23-27, wherein an activity of the multi-gene circuit comprises expression of 1, 2, 3, 4, or more polypeptides encoded by the gene components.

29. The SMC of claim 25, wherein the activator is a small molecule and optionally is soluble.

30. The SMC of claim 25, wherein the activator is present on or proximal to a disease site within the body.

31. The SMC of any one of claims 20-30, wherein an activity of a first multi-gene genetic circuit modulates an activity of at least one additional multi-gene genetic circuit, and optionally activates a cascade of activity of 1, 2, 3, 4, or more additional gene components of the multi-gene genetic circuit in the SMC or in at least one additional SMC.

32. The SMC of any one of claims 20-30, wherein the SMC comprises one or more of prokaryotic or eukaryotic transcription/translation (TX/TL) components.

33. The SMC of any one of claims 20-30, wherein the SMC comprises one or more expression vectors comprising one or more of the gene components.

34. The SMC of claim 33, wherein the expression vector comprises one or more of: a promoter sequence and a polynucleotide sequence encoding a polypeptide.

35. The SMC of claim 34, wherein the polynucleotide sequence encodes at least one of a membrane channel polypeptide and a detectable label polypeptide.

36. The SMC of any one of claims 20-30, wherein the SMC comprises a fusion-inducing polypeptide in association with the SMC's exterior surface.

37. The SMC of claim 36, wherein the fusion-inducing polypeptide is a SNARE polypeptide or a SNARE polypeptide mimic.

38. The SMC of any one of claims 20-30, wherein the SMC is fused to at least a second SMC comprising at least one independently selected multi-gene genetic circuit.

39. The SMC of claim 38, wherein the SMC and the second SMC comprise the independently selected multi-gene genetic circuit.

40. The SMC of claim 38, wherein the SMC does not comprise the independently selected multi-gene genetic circuit of the second SMC.

41. A composition comprising a plurality of the SMCs of any one of claims 1-40, wherein the multi-gene genetic circuits of the SCMs are independently selected.

42. The composition of claim 41, wherein the multi-gene genetic circuit of the SMCs comprises 1, 2, 3, 4, or more independently selected gene components.

43. The composition of claim 41 or 42, wherein the SMCs in the plurality comprise the same multi-gene genetic circuit.

44. The composition of claim 41 or 42, wherein the SMCs in the plurality comprise independently selected multi-gene genetic circuits.

45. The composition of any one of claims 40-44, wherein contacting an SMC of the plurality of SMCs with an externally delivered agent modulates an activity of at least one gene component of the multi-gene genetic circuit of the contacted SMC.

46. The composition of any one of claims 41-44, wherein at least one of the SMCs in the plurality of SMCs is fused to another of the SMCs in the plurality of SMCs.

47. The composition of any one of claims 41-43, wherein one or more multi-gene genetic circuits in two or more SMCs of the plurality of SMCs are active in parallel.

48. The composition of any one of claims 41-45, wherein an activity of one or more multi-gene genetic circuits in a first SMC of the plurality is modulated by at least one of: (1) an activity of a multi-gene genetic circuit in the first SMC of the plurality; and (2) an activity of a multi-gene genetic circuit in a second SMC of the plurality.

49. The composition of any one of claims 41-48, wherein two or more of the plurality of SMCs operate in conjunction with each other as a network.

50. The composition of claim 49, wherein operating in conjunction with each other comprises being in chemical communication with each other.

51. The composition of any one of claims 39-48, wherein an activity of the multi-gene genetic circuit comprises expression of 1, 2, 3, 4, or more polypeptides.

52. The composition of any one of claims 39-49, wherein an activity of a first gene component of an SMC of the plurality of SMCs modulates an activity of one or more additional gene components of at least one of: (1) the multi-gene genetic circuit of the SMC and (2) a multi-gene genetic circuit of another SMC of the plurality of SMCs.

53. The composition of claim 50, wherein the multi-gene genetic circuit of (2) is different than the multi-gene genetic circuit of (1).

54. The composition of any one of claims 39-51, wherein an activity of a first multi-gene genetic circuit of an SMC of the plurality of SMCs modulates an activity of at least one additional multi-gene genetic circuit of an SMC of the plurality of SMCs, and optionally activates a cascade of activity of 1, 2, 3, 4, or more additional gene components of the first multi-gene genetic circuit in the SMC or in at least one additional SMC in the plurality of SMCs.

55. The composition of any one of claims 39-52, wherein the plurality of SMCs comprises one or more of: bacterial transcription/translation (TX/TL) components and mammalian TX/TL components.

56. The composition of any one of claims 39-66, wherein the plurality of SMCs comprises one or more independently selected expression vectors.

57. The composition of claim 54, wherein the expression vector comprises one or more of: a promoter sequence and a polypeptide-encoding polynucleotide sequence.

58. The composition of claim 55, wherein the polynucleotide sequence encodes at least one of: a membrane channel polypeptide and a detectable label polypeptide.

59. The composition of any one of claims 39-56, wherein at least a portion of the SMCs in the plurality of SMCs comprise a fusion-inducing polypeptide in association with the SMCs' exterior surfaces.

60. The composition of claim 57, wherein the fusion-inducing polypeptide is a SNARE polypeptide or a SNARE polypeptide mimic.

61. The composition of claim 58, wherein the SNARE polypeptide or SNARE polypeptide mimic associated with the exterior surface of the SMCs in a first portion of the plurality of SMCs that comprise a fusion-inducing polypeptide, is complementary to the SNARE polypeptide or SNARE polypeptide mimic associated with the exterior surface of the SMCs in a second portion of the plurality of SMCs.

62. The composition of any one of claims 39-60, wherein an activity of a first multi-gene genetic circuit in one or more SMCs of the plurality of SMCs activates at least one additional multi-gene genetic circuit in one or more SMCs of the plurality of SMCs.

63. The composition of claim 60, wherein an activity of a multi-gene genetic circuit in an SMC of the plurality of SMCs results in a cascade of multi-gene genetic circuit activation in one or more SMCs of the plurality of SMCs.

64. The composition of any one of claims 39-61, wherein an activity of a first multi-gene genetic circuit in a first SMC of the plurality of SMCs activates 1, 2, 3, 4, or more additional multi-gene genetic circuits in one or more of: (1) the first SMC and (2) a second SMC of the plurality of SMCs.

65. The composition of claim 62, wherein the additional multi-gene genetic circuit is selected from: (1) a genetic circuit that is the same as the first multi-gene genetic circuit and (2) a multi-gene genetic circuit that is different than the first multi-gene genetic circuit.

66. The composition of any one of claims 39-63, wherein contacting at least one SMC of the plurality of SMCs with an externally delivered agent modulates an activity of at least one of the multi-gene genetic circuits of the contacted SMC.

67. The composition of any one of claims 39-64, wherein an activity of a multi-gene genetic circuit of an SMC of the plurality of SMCs results in contacting one or more multi-gene genetic circuits of the SMC with an agent that modulates an activity of the one or more multi-gene genetic circuits.

68. A method for treating a condition or disease that benefits from a therapeutic agent comprising administering to a subject in need thereof a SMC of claim 1 or claim 2.

69. The method of claim 68 wherein the SMC targets a site of disease.

70. The method of claim 68 wherein the disease is cancer, thrombosis or enzyme deficiency.

71. The method of claim 68 wherein the treating comprises:
(a) administering a SMC comprising a sensor that detects an externally applied signal; and
(b) applying the signal to the part of the body in which delivery of the therapeutic agent is desired.

72. The method of claim 68 wherein the therapeutic agent is a protein, peptide, nucleic acid or small molecule.

73. The method of claim 68 wherein the nucleic acid is DNA, RNA, shRNA, siRNA, an antisense oligonucleotide, a microRNA inhibitor, an anti-miRNA or a sgRNA.

74. The method of claim 68 wherein the protein is an antibody, toxin, hormone, marker or an enzyme.

75. The method of claim 68 wherein the peptide is an antibiotic or antimicrobial peptide.

76. A method for diagnosing a condition or disease comprising administering to a subject in need thereof a SMC of claim 1 or claim 2.

77. The method of claim 76 wherein the SMC targets a site of disease.

78. The method of claim 76 wherein the disease is cancer, thrombosis or enzyme deficiency.

79. The method of claim 6 wherein the diagnosing comprises:
(a) administering a SMC comprising a sensor that detects an externally applied signal; and
(b) applying the signal to the part of the body in which diagnosis of the disease is desired.

80. The method of claim 76 wherein the diagnostic agent is a protein, peptide, nucleic acid or small molecule.

81. The method of claim 76 wherein the nucleic acid is DNA, RNA, shRNA, siRNA, an antisense oligonucleotide, a microRNA inhibitor, an anti-miRNA or a sgRNA.

82. The method of claim 76 wherein the protein is an antibody, toxin, hormone, marker or an enzyme.

83. The method of claim 76 wherein the peptide is an antibiotic or antimicrobial peptide.

84. The method of claim 76 wherein the diagnostic agent is detected in a bodily fluid of the subject.

85. The method of claim 84 wherein the bodily fluid is blood, urine, saliva, cerebrospinal fluid, ascites, or lymphatic fluid.

86. The method of claim 76 wherein the diagnostic agent is detected by imaging.
